(19) United States
(12) Patent Application Publication
(10) Pub. No.: US 2020/0078183 A1
(43) Pub. Date: Mar. 12, 2020

(54) NON-MODULAR, STEMMED, FEMORAL COMPONENT AND METHOD FOR TOTAL KNEE ARTHROPLASTY

(71) Applicant: Adam Rosen, San Diego, CA (US)

(72) Inventor: Adam Rosen, San Diego, CA (US)

(21) Appl. No.: 16/542,031

(22) Filed: Aug. 15, 2019

Related U.S. Application Data
(60) Provisional application No. 62/730,483, filed on Sep. 12, 2018.

(51) Int. Cl. A61F 2/38

(52) U.S. Cl. CPC A61F 2/3859; A61F 2002/30574; A61F 2002/3863

(57) ABSTRACT

A non-modular, stemmed, femoral component for setting of a femur in total knee arthroplasty to obtain additional support and fixation, the femur including an anterior flange, an anterior cortex, a medullary canal, and an intercondylar notch, comprising a femoral component including a back; a non-modular stem fixed to the back of the femoral component and configured to be in line with the medullary canal, posterior to the anterior flange to allow space for the anterior cortex, and anterior to the intercondylar notch to prevent interruption of insertion of a posterior cruciate ligament, if the posterior cruciate ligament is to be maintained, when applied to the femur during setting of a femur in total knee arthroplasty.

### CROSS REFERENCE TO RELATED APPLICATION

This application claims the benefit of U.S. Provisional Patent Application No. 62/730,483, filed on Sep. 12, 2018, under 35 U.S.C. 119. This Provisional Patent Application is incorporated by reference herein.

### FIELD OF THE INVENTION

The present invention relates to a non-modular, stemmed, femoral component for total knee arthroplasty.

### BACKGROUND OF THE INVENTION

Total knee replacements (TKR) are a common and successful surgery for the treatment of knee arthritis. Currently there are many different companies, all with similar designs and styles to treat the varied anatomy of patients.

Most commonly TKRs are performed using a solid metal tibial tray, or component, which is placed on the tibia, or shin bone, in which a plastic insert is placed. Additionally, a femoral component such as that shown in FIG. 2A is placed on the femur, or thigh bone.

In revision total knee replacements (RTKRs) the tibial and femoral components are modular, and one can add stems to each component, allowing the implant to be fixed not only to the end of the bone, but anchored within the bone for added stability. FIG. 2B shows a prior art modular femoral component design including the femoral component, an adapter bushing, and a stem that are added to the component.

There are patient populations that fit between the spectrum of the standard TKR and the revision group. These groups of patients undergo a surgery which has commonly been referred to as the complex total knee replacement (CTKR). Typically, these are elderly patients with poor bone quality or obese patients which are at greater risk for implant failure. Sometimes intraoperative complications occur, such as a fracture, or break, in the femur, that would require additional fixation to prevent implant failure. In these high-risk patients, surgeons typically use a standard femoral component but add a stem to a modular tibial component to gain better fixation in the tibia. This adds cost to the procedure and time to perform the additional steps, but the added steps are minimal as one only has to ream, or prepare the bone in the tibia, with a straight reamer (large drill bit).

On the femoral side adding a stem is more complicated. To stem the femoral side additional bone cuts need to be made, requiring more time. Additional sets of instruments from the company are required, and additional banks of implants are needed to build a modular, stemmed femoral implant, which may not be available. Another problem is that the junctions between the femoral component, adapter bushing, and stem are susceptible to failing/breaking/fretting. These limitations result in many surgeons forgoing this step and to accept a routine, or primary, femoral component, without the added support of a stem. This could result in early failure of the TKR.

### SUMMARY OF THE INVENTION

An aspect of the invention is a non-modular, stemmed, femoral component that bridges the gap between standard femoral implants and revision, modular, stemmed femoral implants. The invention would accommodate most patients in need of a stem on the femoral component when performing a CTKR. The non-modular, stemmed femoral component fills a void for CTKR between the standard TKR implants and RTKR.

Another aspect of the invention involves a non-modular, stemmed, femoral component for setting of a femur in total knee arthroplasty to obtain additional support and fixation, the femur including an anterior flange, an anterior cortex, a medullary canal, and an intercondylar notch, comprising a femoral component including a back; a non-modular stem fixed to the back of the femoral component and configured to be in line with the medullary canal, posterior to the anterior flange to allow space for the anterior cortex, and anterior to the intercondylar notch to prevent interruption of insertion of a posterior cruciate ligament, if the posterior cruciate ligament is to be maintained, when applied to the femur during setting of a femur in total knee arthroplasty.

One or more implementations of the aspect of the invention described immediately above includes one or more of the following: the non-modular, stemmed, femoral component is a kit of non-modular, stemmed, femoral components with the non-modular, stemmed, femoral components including respective non-modular stems with a length and a diameter that varies anthropometrically from a smallest size up to a largest size; and/or the stem includes a tip and the stem ranges in length from 50 mm to 100 mm so that the tip of the stem bypasses a most proximal aspect of the anterior flange.

A further aspect of the invention involves a method of using the non-modular, stemmed, femoral component of the aspect of the invention described immediately above, comprising setting the femur in total knee arthroplasty to obtain additional support and fixation by aligning the non-modular stem with the medullary canal, posterior to the anterior flange to allow space for the anterior cortex, and anterior to the intercondylar notch to prevent interruption of insertion of a posterior cruciate ligament, if the posterior cruciate ligament is to be maintained.

One or more implementations of the aspect of the invention described immediately above includes one or more of the following: the non-modular, stemmed, femoral component is a kit of non-modular, stemmed, femoral components with the non-modular, stemmed, femoral components including respective non-modular stems with a length and a diameter that varies anthropometrically from a smallest size up to a largest size, and the method further comprising sizing a variety of the non-modular, stemmed, femoral components of the kit of non-modular, stemmed, femoral components to determining the most appropriate non-modular, stemmed, femoral component to be used during setting of the femur in total knee arthroplasty; and/or the stem includes a tip and the stem ranges in length from 50 mm to 100 mm, and sizing the variety of the non-modular, stemmed, femoral components includes determining if the tip of the stem bypasses a most proximal aspect of the anterior flange.

### BRIEF DESCRIPTION OF DRAWINGS

The accompanying drawings, which are incorporated in and form a part of this specification illustrate embodiments of the invention and together with the description, serve to explain the principles of the invention.

FIG. 1 is a front elevational view of an embodiment of a non-modular femoral component on the left with two prior-art total knee designs shown to the right.

FIG. 2A is a side elevational view of a prior art femoral component design without any stem attachment.

FIG. 2B is a side elevational view of a prior art modular femoral component design showing that stem can be added to the component.

FIG. 3 is a side elevational view of the non-modular femoral component shown in FIG. 1.

FIG. 4 is a perspective view of the non-modular femoral component shown in FIG. 1.

FIG. 5 is another perspective view of the non-modular femoral component shown in FIG. 1.

FIG. 6 is a further perspective view of the non-modular femoral component shown in FIG. 1.

FIG. 7 shows the non-modular femoral component applied to an end of the femur in femur and tibia model.

### DESCRIPTION OF EMBODIMENT OF THE INVENTION

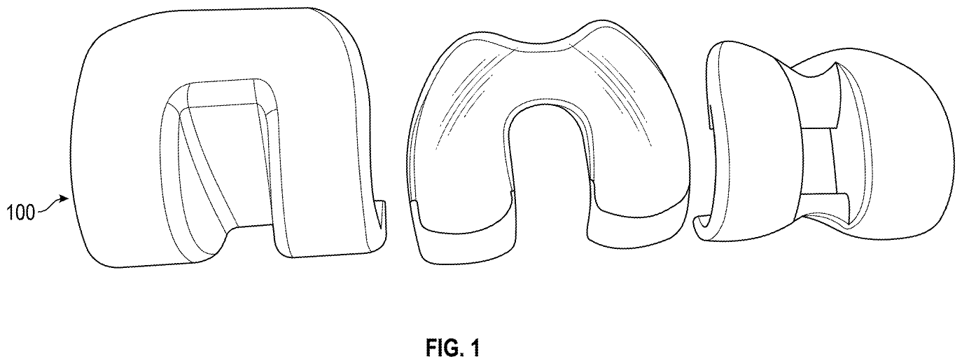

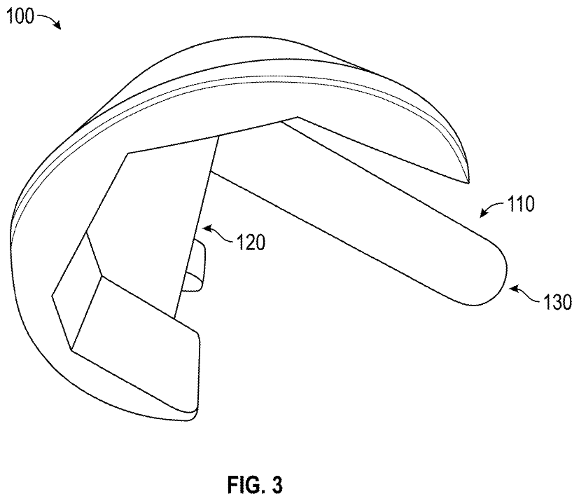

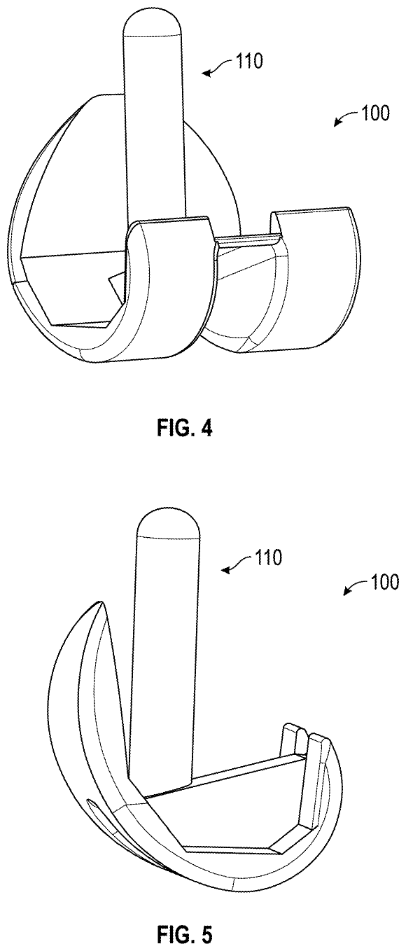

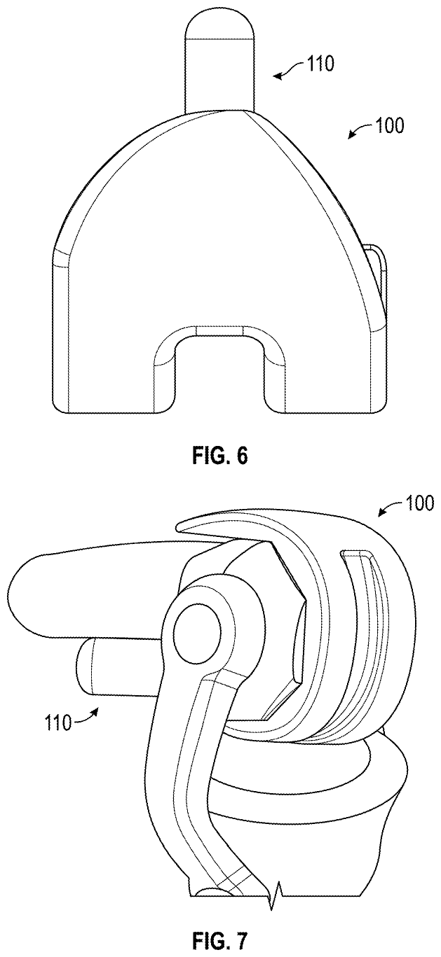

With reference to FIGS. 1 and 3-7, an embodiment of a stemmed, non-modular femoral component (“femoral component”) 100 for total knee arthroplasty will be described.

The femoral component 100 includes a non-modular stem 110. The stem 110 is fixed to a back 120 of the femoral component 100 and is configured to be in line with the medullary canal in use. Anthropometric sizing allows the stem 110 to be posterior to the anterior flange to allow space for the anterior cortex of the femur and anterior to the intercondylar notch to prevent interruption of the insertion of the posterior cruciate ligament, if it is to be maintained. In knees where the posterior cruciate ligament is sacrificed, the box will accommodate the appropriate tibial plastic insert. The stem length and diameter would grow anthropometrically from the smallest sizes up to the largest sizes. The stem 110 would range in length from 50 mm to 100 mm to allow for a tip 130 of the stem 110 to bypass the most proximal aspect of the anterior flange.

The femoral component 100 works in the same way current femoral components work in the setting of total knee arthroplasty, except that the femoral component 100 allows a surgeon the intraoperative flexibility to use the femoral component 100 with the stem 110 to obtain additional support and fixation.

The femoral component 100 allows a surgeon additional intra-operative flexibility in performing CTKRs. Instances where a stem would be useful are when iatrogenic notching occurs during the case, which increases the risk of fracture. Obesity is a growing epidemic and many surgeons have stemmed the tibia for fear of loosening due to the excess weight placed on the implant. The stemmed femoral component 100 offers the same advantages without having to go to a more expensive and complex revision component. As the population ages, more patients with osteoporosis are undergoing TKA. Stemmed components may be of benefit to decrease the risk of peri-prosthetic fractures.

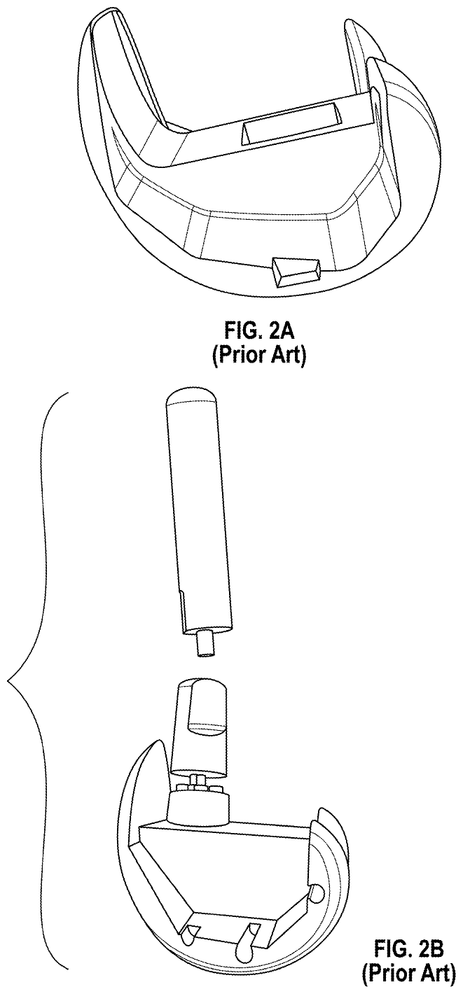

Currently surgeons have limited options if there was inadequate bone quality or concern of the quality of the bone necessitating a stem; surgeons would have to switch to a revision system. This requires multiple additional steps, trays, and implants. This also adds significant cost as revision implants are significantly more expensive than primary implants. In this era of cost containment, the non-modular, stemmed femoral component 100 fills a void that would potentially decrease the cost to the hospitals, patients and society. An additional problem with the revision system, as shown in FIG. 2B and mentioned above, is that the junctions between the femoral component, adapter bushing, and stem are susceptible to failing/breaking/fretting.

The other problem surgeons run into is the unexpected need for a stem on the femoral side during a surgical case. Most hospitals do not keep the revision systems within the hospital but have those implants and instruments brought in on a case per case basis when it is anticipated to be needed. This may require numerus trays of instruments and boxes of implants. Having a back-up bank of stemmed femoral components 100 allows the surgeon to deal with intra-operative issues should they arise. If unexpected problems occur during a case, the surgeon may not have all of the revision system and instrumentation available.

The non-modular, stemmed femoral component 100 overcomes the problems discussed above and may be provided as a simple back-up bank of implants (a select group of sizes for right and left femurs) with minimal instrumentation.

The above figures may depict exemplary configurations for the invention, which is done to aid in understanding the features and functionality that can be included in the invention. The invention is not restricted to the illustrated architectures or configurations, but can be implemented using a variety of alternative architectures and configurations. Additionally, although the invention is described above in terms of various exemplary embodiments and implementations, it should be understood that the various features and functionality described in one or more of the individual embodiments with which they are described, but instead can be applied, alone or in some combination, to one or more of the other embodiments of the invention, whether or not such embodiments are described and whether or not such features are presented as being a part of a described embodiment. Thus the breadth and scope of the present invention, especially in the following claims, should not be limited by any of the above-described exemplary embodiments.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

I claim:

1. A non-modular, stemmed, femoral component for setting of a femur in total knee arthroplasty to obtain additional support and fixation, the femur including an anterior flange, an anterior cortex, a medullary canal, and an intercondylar notch, comprising:
a femoral component including a back;
a non-modular stem fixed to the back of the femoral component and configured to be in line with the medullary canal, posterior to the anterior flange to allow space for the anterior cortex, and anterior to the intercondylar notch to prevent interruption of insertion of a posterior cruciate ligament, if the posterior cruciate ligament is to be maintained, when applied to the femur during setting of a femur in total knee arthroplasty.

2. The non-modular, stemmed, femoral component of claim 1, wherein the non-modular, stemmed, femoral component is a kit of non-modular, stemmed, femoral components with the non-modular, stemmed, femoral components including respective non-modular stems with a length and a diameter that varies anthropometrically from a smallest size up to a largest size.

3. The non-modular, stemmed, femoral component of claim 2, wherein the stem includes a tip and the stem ranges in length from 50 mm to 100 mm so that the tip of the stem bypasses a most proximal aspect of the anterior flange.

4. A method of using the non-modular, stemmed, femoral component of claim 1, comprising setting the femur in total knee arthroplasty to obtain additional support and fixation by aligning the non-modular stem with the medullary canal, posterior to the anterior flange to allow space for the anterior cortex, and anterior to the intercondylar notch to prevent interruption of insertion of a posterior cruciate ligament, if the posterior cruciate ligament is to be maintained.

5. The method of using the non-modular, stemmed, femoral component of claim 4, wherein the non-modular, stemmed, femoral component is a kit of non-modular, stemmed, femoral components with the non-modular, stemmed, femoral components including respective non-modular stems with a length and a diameter that varies anthropometrically from a smallest size up to a largest size, and the method further comprising sizing a variety of the non-modular, stemmed, femoral components of the kit of non-modular, stemmed, femoral components to determining the most appropriate non-modular, stemmed, femoral component to be used during setting of the femur in total knee arthroplasty.

6. The method of using the non-modular, stemmed, femoral component of claim 5, wherein the stem includes a tip and the stem ranges in length from 50 mm to 100 mm, and sizing the variety of the non-modular, stemmed, femoral components includes determining if the tip of the stem bypasses a most proximal aspect of the anterior flange.
